(19) United States
(12) Patent Application Publication
(10) Pub. No.: US 2014/0264205 A1
(43) Pub. Date: Sep. 18, 2014

(54) SYSTEMS AND METHODS FOR RECHARGING AN AUTO-INJECTOR

(71) Applicants: Morris Elijah Worden, New York, NY (US); Anthony Torris, Montclair, NJ (US); Alistair Bramley, Brooklyn, NY (US); Eric Freitag, Brooklyn, NY (US); Daniel Formosa, Piermont, NY (US)

(72) Inventors: Morris Elijah Worden, New York, NY (US); Anthony Torris, Montclair, NJ (US); Alistair Bramley, Brooklyn, NY (US); Eric Freitag, Brooklyn, NY (US); Daniel Formosa, Piermont, NY (US)

(21) Appl. No.: 13/835,407

(22) Filed: Mar. 15, 2013

(51) Int. Cl. B66F 19/00; B23P 6/00

(52) U.S. Cl. CPC B66F 19/00; B23P 6/00
USPC 254/133 A; 29/402.01

(57) ABSTRACT

Systems, devices, and methods are described for resetting an auto-injector. In one aspect, a device for resetting an auto-injector comprises a housing having a receiving shaft into which the auto-injector can be inserted in a receiving direction, a reset element configured to engage a collar of the auto-injector, and a post configured to engage a plunger within the auto-injector when the auto-injector is inserted into the receiving shaft. The reset element moves the collar relative to the auto-injector in a first direction along the receiving direction. The post moves the plunger in a second direction relative to the auto-injector opposite the receiving direction.

### BACKGROUND

Auto-injectors are devices used for administering medication. Auto-injectors are designed for ease of use for self-administration by patients, as well as for administration by others who may lack medical training. In some designs, the dose can be administered by placing the auto-injector at the site of injection and then pressing a button that causes a syringe needle to automatically inject the drug. In other designs, the dose can be administered by pushing the auto-injector against the site of injector in a single motion.

Some auto-injectors are designed to be reusable and can be reset (“recharged”) after use. Recharging an auto-injector requires replacing the drug, as well as manipulating the auto-injector to reset the position of the plunger and various other components that may need to be reset. For patients with limited manual dexterity, this can be impractical and tedious, particularly if the user has unsteady hands. Such methods can also be tedious in situations in which the auto-injector functionality is to be demonstrated, which may require that the demonstrator actuates the auto-injector numerous times. It would be desirable to provide a simpler way for recharging an auto-injector. It would also be desirable if the user could rest the auto-injector in a single motion.

### SUMMARY

Disclosed herein are systems, devices, and methods for resetting an auto-injector. In one aspect, a device for resetting an auto-injector comprises a housing having a receiving shaft into which the auto-injector can be inserted in a receiving direction, a reset element configured to engage a collar of the auto-injector when the auto-injector is inserted into the receiving shaft, which moves the collar relative to the auto-injector in a first direction along the receiving direction, and a post configured to engage a plunger within the auto-injector when the auto-injector is inserted into the receiving shaft, which moves the plunger in a second direction relative to the auto-injector opposite the receiving direction.

In some implementations, the reset element comprises a frame, and at least two elongate members extending from the frame and defining an opening therebetween. An inner ledge may extend along the frame and the at least two elongate members about the opening, and may be configured to couple with the collar. The portion of the inner ledge may be tapered to form a wedge.

In some implementations, the device further comprises a base, and a track. The post and the track may be connected to the base, and the reset element may engage and be displaced along the track.

In some implementations, the reset element may move the collar relative to the auto-injector as the reset element is displaced along a first portion of the track. The reset element may fix the position of the collar relative to the auto-injector as the reset element is displaced along a second portion of the track different than the first portion of the track. A plane defined by a portion of the reset element that engages the collar may remain normal to the receiving direction as the reset element is displaced along the track.

In some implementations, the housing may be configured to move relative to the base along the receiving direction in response to the reset element engaging the collar.

In some implementations, the post may be shaped to fit a complementary feature on the plunger.

In one aspect, a method for resetting an auto-injector comprises receiving the auto-injector in a receiving direction, engaging a collar of the auto-injector with a reset element, which moves the collar relative to the auto-injector in the receiving direction, and engaging a plunger within the auto-injector with a post, which moves the plunger relative to the auto-injector opposite the receiving direction.

In some implementations, the reset element may be displaced along a track, and may move the collar relative to the auto-injector is displaced along a first portion of the track. The reset element may fix the position of the collar relative to the auto-injector as the reset element is displaced along a second portion of the track. A plane defined by a portion of the reset element that engages the collar may remain normal to the receiving direction as the reset element is displaced along the track.

In some implementations, engaging the plunger with the post comprises fitting a portion of the post to a complementary feature on the plunger.

In one aspect, a device for resetting an auto-injector comprises means for receiving the auto-injector in a receiving direction, a first reset means for engaging a collar of the auto-injector, which moves the collar relative to the auto-injector in the receiving direction, and a second reset means for engaging a plunger within the auto-injector, which moves the plunger relative to the auto-injector opposite the receiving direction.

In some implementations, the first reset means comprises a frame portion, at least two elongate members extending from the frame portion defining an opening therebetween, and coupling means that extend along at least a portion of the frame portion and the elongate members about the opening, where the coupling means is configured to couple with the collar. A portion of the inner ledge may be tapered to form a wedge.

In some implementations, the device comprises a supporting means and a track. The second reset means and the track may be connected to the supporting means, and the first reset means may be displaced along the track.

In some implementations, the track may comprise a first portion and a second portion. The first reset means may move the collar relative to the auto-injector as the first reset means is displaced along the first portion of the track. The reset element may fix the position of the collar relative to the auto-injector as the reset element is displaced along the second portion of the track.

In some implementations, the device further comprises means for orienting the first reset means such that a plane defined by a portion of the reset means that engages the collar remains normal to the receiving direction as the first reset means is displaced along the track.

In some implementations, the device further comprises housing means configured to move relative to the supporting means along the receiving direction in response to the first reset means engaging the collar.

In some implementations, the second reset means may be shaped to fit a complementary feature on the plunger.

In one aspect, a reset element comprises a frame portion and at least two elongate members extending from the frame portion and defining a first opening therebetween, where an inner ledge extends along at least a portion of the frame portion and the elongate members about the opening, and the inner ledge is configured to couple with an auto-injector.

In some implementations, a portion of the inner ledge may be tapered to form a wedge. The first opening is defined by the elongate members may have a horseshoe shape.

In some implementations, the frame defines a second opening that extends from a top portion of the frame to a bottom portion of the frame, and at least one tracking pin inside the opening and directed inward that is configured to engage a separate track that passes through the opening. The reset element may include at least one tracking pin that extends outward from the frame that is configured to engage a separate track. The tracking pins may be cylindrically shaped.

In one aspect, a reset element comprises a frame portion and coupling means that extend from the frame portion and define an opening, and are configured to couple with the collar.

In some implementations, the coupling means comprises an inner ledge that is tapered to form a wedge. The opening may be horseshoe shaped.

In some implementations, the frame further comprises means for engaging a separate track. The means for engaging the separate track may include a tracking pin, which may extend outward from the frame and is configured to engage a separate track. The tracking pin may be cylindrically shaped.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIGS. 1A-1C show cross-sectional views of an illustrative recharge device system engaging an auto-injector at various recharge positions;

FIG. 2A shows a perspective view of an illustrative auto-injector that has not been actuated;

FIG. 2B shows a perspective view of an illustrative auto-injector that has been actuated;

FIG. 3 shows a perspective view of an illustrative recharge device;

FIG. 3A shows an auto-injector being inserted by a user into the recharge device of FIG. 3;

FIG. 3B shows the auto-injector of FIG. 2A being depressed to reset the auto-injector;

FIG. 3C shows the auto-injector of FIG. 2B after it has been removed from the recharge device of FIG. 3;

FIG. 4 shows an exploded perspective view of an illustrative recharge device;

FIG. 5 shows a perspective view of an illustrative reset element used for resetting an auto-injector;

FIGS. 6A and 6B show perspective partial cross-sectional views of an illustrative reset element constrained to various positions in a housing of the recharge device; and

FIGS. 7A-7C show perspective views of an illustrative reset element positioned along a track at various positions.

### DETAILED DESCRIPTION

To provide an overall understanding of the systems, devices, and methods described herein, certain illustrative embodiments will be described. Although the embodiments and features described herein are specifically described for use in connection with resetting an auto-injector, it will be understood that all the components, connection mechanisms, adjustable systems, and other features outlined below may be combined with one another in any suitable manner and may be adapted and applied to other mechanical devices that may require simultaneous adjustment and resetting of movable components.

The systems, devices, and methods described herein for recharging an auto-injector may be used for any suitable auto-injector, including, for example, the auto-injector system described in U.S. patent application Ser. No. [______] (Attorney Docket No. 106471-0012-101), filed concurrently herewith, and entitled “SYSTEMS AND METHODS FOR DAMPENING FRICTION IN AN AUTOINJECTOR DEVICE,” the disclosure of which is hereby incorporated by reference herein in its entirety.

FIGS. 1A-1C show a cross-sectional view of the components of a recharge device 100 engaging the components of an auto-injector 200 at various positions of the auto-injector 200 within the recharge device 100. The auto-injector 200 shown may have, for example, a handle 210, a plunger 220 located inside the auto-injector 200, a firing spring 222 configured to fire the plunger 220, and a collar 230 for visually indicating whether the auto-injector 200 has been actuated. The recharge device 100 shown is configured to receive the auto-injector 200 through a receiving shaft 110, located within a carriage housing 120. The receiving shaft 110 shown is cylindrical for receiving a cylindrical auto-injector, however any suitable shape may be used to accommodate auto-injectors of varying shapes. The carriage housing 120 is configured to move relative to an external housing 150 with respect to a longitudinal axis 130. The external housing 150 is connected to a base unit 140 that serves as a structural support for the components of the recharge device 100. The base unit 140 has a post 142 that extends along the longitudinal axis 130. In certain embodiments, the base unit 140 has a track 144 along which a reset element 300 travels as the carriage housing 120 is moved relative to the base unit 140. In certain embodiments, the track 144 is structured for engaging a portion of the reset element 300 such that the movement of the reset element 300 is at least partially guided by the track 144. The carriage housing 120 has a spring port 122 that contains a compression spring 124. In some embodiments, one or more compression springs may be positioned at any suitable location within recharge device 100. For example, a compression spring may surround the post 142 and may abut a bottom surface of the carriage housing. The compression spring 124 engages the base unit 140 to maintain a default relative position, or biased position, between the carriage housing 120 and the base unit 140, as shown in FIG. 1A. For example, if a force is applied to the carriage housing 120 along the longitudinal axis 130 toward the base unit 140, the compression spring 124 may provide a restoring force in the opposite direction. The base unit 140 may be connected to the external housing 150 such that the external housing 150 partially contains the carriage housing 120 and reset element 300, and completely contains the post 142 and track 144.

The auto-injector 200 is received by the recharge device 100 in a receiving direction 132 that runs parallel to the longitudinal axis 130. The auto-injector 200 can be received, for example, by a user grasping the handle 210 of the auto-injector 200 and inserting it into the recharge device 100 along the receiving direction 132. The reset element 300 is configured to engage the collar 230 of the auto-injector 200, as shown in FIG. 2A. FIG. 1A shows the auto-injector 200 after it has been received by the receiving shaft 110, prior to being engaged by the reset element 300. If the user inserts the auto-injector 200 into the receiving shaft 110 such that it is fully-received, the auto-injector 200 may remain in a resting position, as shown in FIG. 1A. At this point, the user may then apply a force to the handle 210 of the auto-injector 200 in the receiving direction 132, which results in the reset element 300 engaging the collar 230 of the auto-injector 200.

The auto-injector 200 will then reach the intermediate position shown in FIG. 1B. At this point, the reset element 300 fully engages the collar 230 of the auto-injector 200. The reset element 300 is configured to move the collar 230 of the auto-injector 200 along the receiving direction 132, extending the collar 230 away from the body of the auto-injector 200 until it is in a reset position. The reset element 300 keeps the collar 230 in the reset position as the auto-injector 200 is further pushed in the receiving direction 132 by the user. The reset element 300 moves along the receiving direction 132 as the auto-injector 200 is pushed by the user in the receiving direction 132. In certain embodiments, the carriage housing 120 is configured to move along the receiving direction 132 towards the base unit 140 as the auto-injector 200 is pushed in the receiving direction 132. For example, the reset element 300 may be housed within the carriage housing 120 to partially direct its movement with respect to the auto-injector 200. When the auto-injector 200 reaches the position shown in FIG. 1B, the post 142 within the recharge device 100 engages the plunger 220 within the auto-injector 200. In certain embodiments, the end of the post 142 is shaped to engage with a complementary feature on the plunger 220. For example, if the plunger 220 has an indented region at its end, the post 142 may have a protruding region that fits within the indented region of the plunger 220.

FIG. 1C shows the distal position of the auto-injector 200 within the recharge device 100 in response to the user continuing to apply a force to the handle 210 of the auto-injector 200. As the auto-injector 200 is moved from the intermediate position of FIG. 1B to the distal position of FIG. 1C, the post 142 moves the plunger 220 in a direction opposite the receiving direction 132 with respect to the auto-injector 200. FIG. 1C shows the plunger 220 reset to a reset position within the auto-injector 200. In response to the plunger 220 reaching the reset position, the user may receive haptic feedback from the auto-injector 200 indicating that the plunger 220 has been reset. For example, the auto-injector may produce a clicking sound upon the plunger 220 returning to its reset position. At this point, the user may allow the compression spring 124 to provide a restoring force that moves the carriage housing 120 along the longitudinal axis 130 opposite the receiving direction 132 in order to restore the carriage housing 120 to its original position, as shown in FIG. 1A. This may also allow the reset element 300 and the auto-injector 200 to return to their respective positions, as shown in FIG. 1A. The auto-injector 200 may now be reset to its reset configuration in which both the collar 230 and plunger 220 are in their respective reset positions. At this point, the user may retrieve the auto-injector 200 by grasping the handle 210 and removing it from the receiving shaft 110.

In certain embodiments, the process illustrated in FIGS. 1A-1C is automated. For example, upon receiving the auto-injector 200 into the receiving shaft 110, the carriage housing 120 may be actuated by a motor within the recharge device 100 that moves the carriage housing 120 toward and away from the base unit 140. As the carriage housing 120 moves toward the base unit 140, the auto-injector 200 is moved toward the base unit 140 where it is then engaged by the reset element 300. The reset element 300 engages the collar 230 of the auto-injector 200 such that the auto-injector 200 is locked within the carriage housing 120. Locking the auto-injector 200 in this way prevents the auto-injector 200 from being dislodged from the receiving shaft 110 when the post 142 engages the plunger 220. The auto-injector 200 is reset at the point in which the carriage housing 120 abuts the base unit 140. The motor may then reverse its direction and actuates the carriage housing 120 in a direction opposite the receiving direction 132. Alternatively, the compression spring 124 may return the carriage housing 120 to its original position shown in FIG. 1A.

FIGS. 2A and 2B show an illustrative auto-injector 200 that is reusable. The auto-injector 200 shown is cylindrical in shape, but may be of any suitable shape. For example, the auto-injector may have a square, rectangular, triangular, or irregular shape. The auto-injector 200 shown in FIG. 2A has a handle 210 for grasping by the user. The handle 210 is designed for ease of use by a user that may have limited manual dexterity, allowing the user to administer injections with one hand. In certain embodiments, the handle 210 is designed to have an ergonomic grip. The auto-injector 200 may have a window 240 that allows the user to access a syringe and drug reservoir within the auto-injector 200. The window 240 may also be used, for example, to load a syringe and drug reservoir after the auto-injector 200 is reset. In certain embodiments, the window 240 is used to show that the plunger 220 has been actuated for demonstration purposes. The auto-injector 200 has a collar 230 that indicates whether the auto-injector 200 has been actuated. In FIG. 2A, the collar 230 is extended away from the body of the auto-injector 200, revealing indicator band 250. When the collar 230 is extended and the plunger 230 is in its reset position, the user can then perform an injection by pressing the auto-injector 200 against the site of injection. FIG. 2B shows the auto-injector 200 after being actuated, in which the collar 230 was pushed inward toward the body of the auto-injector 200 covering up the indicator band 250. When the indicator band 250 is not visible, this indicates to the user that the auto-injector 200 has been actuated and needs to be reset before its next use. In certain embodiments, a component coupled to the plunger 220 may appear in the window 240 when the auto-injector 200 has been actuated. When the indicator band 250 is visible and the component coupled to the plunger is no longer visible in the window 240, this may indicate that the auto-injector 200 has been reset. In certain embodiments, the collar 230 could be replaced by a button that is depressed when the user actuates the auto-injector 200. For example, a depressed button indicates that the auto-injector 200 has been actuated. Resetting the auto-injector 200 returns the button to its non-depressed position, which indicates that the auto-injector 200 is reset. The auto-injector 200 has a syringe shaft 260 through which the syringe delivers the injection. The plunger 220 is accessible through the syringe shaft 260 when the syringe and drug reservoir are removed from the auto-injector 200. In certain embodiments, the syringe and drug reservoir may be loaded into the auto-injector 200 through the syringe shaft 260 rather than the window 240.

FIG. 3 shows an illustrative recharge device 100. The recharge device 100 is supported by the base unit 140, and its internal components are protected and hidden from view by the external housing 150 and carriage housing 120. The carriage housing 120 is extended away from the base unit 140 in its default configuration. The default configuration may be biased by, for example, an internal compression spring 124 that causes the carriage housing 120 to extend distally from the base unit 140. The carriage 112 is located at the top of the carriage housing 120. The receiving shaft 110 extends through the top of the carriage 112 to the interior of the recharge device 100. In certain embodiments, the carriage housing 120 may be colored for aesthetic purposes and to indicate that the recharge device 100 is ready for use. For example, the carriage housing 120 may be the same color as the indicator band 250 of the auto-injector 200. When the carriage housing 120 is no longer visible during the reset process, in accordance with FIG. 1C, this may serve as a visual indicator that the auto-injector 200 has been reset.

FIGS. 3A-3C show a perspective view of the reset process of FIGS. 1A-1C. In FIG. 3A, a user inserts the auto-injector 200 into the receiving shaft 110 of the recharge device 100. If the user does not apply any force to the auto-injector 200, it will rest within the recharge device 100 at the position shown in FIG. 3A. FIG. 3B shows the effect of the user continuing to apply a force to the handle 210 of the auto-injector 200. The carriage housing 120 of the recharge device 100 will track along with the auto-injector 200 relative to the base unit 140 of the recharge device 100, until it is completely hidden by the external housing 150. FIG. 3C shows the user removing the auto-injector 200 from the recharge device 100 after the auto-injector 200 has been reset. As the auto-injector 200 is removed, the carriage housing 120 will return to its default position relative to the base unit 140.

FIG. 4 shows an exploded perspective view of the recharge device 100. The auto-injector 200 is received by the carriage 112. The carriage 112 has an external portion 112a that partially covers the internal components of the recharge device 100, and an internal portion 112b that extends into the carriage housing 120. The receiving shaft 110 passes through the internal portion 112b of the carriage. The carriage 112 meets and connects to the carriage housing 120 at the respective perimeters of the external portion 112a and the top of the carriage housing 120. In certain embodiments, the receiving shaft 110 contains flexible fingers 114, which extend from a lower portion of the receiving shaft 110 to an upper portion of the receiving shaft 110. The flexible fingers 114 serve as bendable cantilevers that conform to an arbitrary shape of an auto-injector. For example, if an auto-injector has an oval shape, flex fingers 114 that contact the widest portion of the auto-injector will flex more than flexible fingers 114 that contact the narrowest portion of the auto-injector, ensuring a tight fit that enables the auto-injector to be received at an arbitrary rotational angle about the receiving direction 132.

The carriage housing 120 contains a distal constraint unit 160, a proximal constraint unit 170, and a reset element 300 that fits within a reset element port 162 within each constraint unit. The distal constraint unit 160 has a carriage port 164 for receiving the internal portion of the carriage 112, and a spring port 122 for the compression spring 124. The distal constraint unit 160 is designed to allow the post 142 to enter inside and engage the auto-injector 200. The distal constraint unit 160 is designed to allow the track 144 to fit inside. The proximal constraint unit 170 is a mirror image of the distal constraint unit 160, allowing both units to come together and fit inside of the carriage housing 120. Once the reset element 300 is constrained to the track 144, the proximal 170 and distal 160 constraint units are brought together to encompass the reset element 300, and the post 142 and track 144 of the base unit 140. The carriage housing 120 is then placed over the constraint units, and the external housing 150 is placed over the carriage housing 120. The bottom portion of the external housing 150 engages and connects to the base unit 140 at the base perimeter. The carriage 112 is then connected to the carriage housing 120 to complete the assembly. In some embodiments, the proximal and distal constraint units 160, 170 may be replaced with a single contiguous unit formed by the carriage housing. For example, the carriage housing may contain a receptacle for holding the reset element 300. The carriage may be configured to couple with the carriage housing such that the reset element 300 is held within the carriage housing and is constrained to slide back and forth within the receptacle.

FIG. 5 shows a perspective view of an illustrative reset element 300. The reset element 300 has a frame 310, a top portion 300a, and a bottom portion 300b. A distal engaging member 320 and a proximal engaging member 330 extend outward from the frame 310, and are configured to fit around and engage an auto-injector 200. The engaging members 320, 330 form an opening 342 that has a generally horseshoe shape for engaging a cylindrical auto-injector 200, although it will be understood that any suitable shape for interfacing with an auto-injector may be used. In certain embodiments, the opening 342 between the engaging members 320, 330 may be shaped to be complementary to the shape of the auto-injector. Located at the top portion 300a of the reset element and in between the distal 320 and proximal 330 engaging members is an inner ledge 340 that protrudes outward into the interior space between the engaging members 320, 330. The inner ledge 340 is tapered as the ledge 340 extends outward along the engaging members 320, 330. The wedge 360 fits between the collar 230 and the body of the auto-injector 200 as the auto-injector 200 is received by the engaging members 320, 330. The wedge 360 pushes the collar 230 away from the body of the auto-injector 200 until the auto-injector 200 is fully engaged by the reset element 300, at which point the collar 230 is returned to its reset position. In certain embodiments, the auto-injector 200 is fully-engaged when the body of the auto-injector 200 abuts an innermost portion of the ledge 340 between the engaging members 320, 330. In other embodiments, the inner ledge 340 may not span the entire perimeter between the engaging members 320, 330, such that the body of the auto-injector 200 abuts the frame 310 when it is fully-engaged. As long as the auto-injector 200 is fully engaged with the reset element 300, the collar 230 will be extended to its reset position. This prevents the collar 230 from being pushed inward toward the body of the auto-injector 200 during the reset process. For example, the collars of some auto-injectors may be coupled to the plunger 220 such that pushing the plunger 220 back into its reset position may also cause the collar 230 to be pushed towards the body of the auto-injector 200. In certain embodiments, the collar 230 of the auto-injector 200 may abut the base unit 140 when it is fully inserted into the recharge device 100. In this case, the wedge 360 will prevent the abutment from pushing the collar 230 towards the body of the auto-injector 200. In some embodiments, the reset element 300 may be used as a standalone device to reset or open the collar 230 of the auto-injector 200. The reset element 300 may be configured to “snap” to the auto-injector 200 once it engages the collar 230 of the auto-injector 200, thereby securing the collar 230 and allowing a user to manually reset the plunger 220.

As shown in FIG. 5, the frame 310 has an opening 370 that extends from the top portion 300a to the bottom portion 300b of the frame, forming a hollow interior for which a track 144 may pass through. The tracking pins 350 are located at the bottom portion 300b of the frame, and are disposed on the interior walls of the opening 370 such that the tracking pins 350 will engage a track 144 that passes through the hollow interior of the frame 310. The tracking pins 350 are cylindrical in shape and are statically fixed to the interior walls, but may be of any suitable shape or structure. For example, the tracking pins 350 may rotate. In certain embodiments, the tracking pins are rotatable gears that engage a track with complementary gear teeth. In certain embodiments, the tracking pins 350, or other suitable structures, are disposed on the exterior walls of the frame 310 and extend outward for engaging with two separate tracks that run along the exterior of the frame 310. In certain embodiments, a single tracking pin may be used. The single tracking pin may be disposed on the interior of the frame 310 to engage a single track that extends through the interior of the frame 310, or it may be disposed on an exterior wall of the frame 310 to engage a single track that runs along the exterior of the frame 310.

FIGS. 6A and 6B show the reset element 300 at different positions within the distal constraint unit 160. The proximal constraint unit 170 is not shown in order to prevent obscuring the view of the reset element 300, and it is assumed that the other components, though not visible, are operating in accordance with the embodiments of the disclosure. FIG. 6A shows the relative location of the reset element 300 with respect to the distal constraint unit 160 corresponding to the configuration shown in FIG. 1A. FIG. 6B shows the relative location of the reset element 300 with respect to the distal constraint unit 160 corresponding to the configuration shown in FIG. 1C. The reset element 300 is positionally- and rotationally-constrained within the reset element port 162 of the distal constraint unit 160 such that it is constrained to move continuously between the two positions shown in FIGS. 5A and 5B. The plane of the top portion 300a of the frame 300 runs parallel to the top interior portion of the reset element port 162 of the distal constraint unit 160, such that the normal of the plane of the top portion 300a remains parallel to the receiving direction 132.

FIGS. 7A-7C show the location of the reset element 300 with respect to the base unit 140 at three different positions during the operation of the recharge device 100, which correspond to FIGS. 1A-1C, respectively. Only the reset element 300 and the base unit 140, with the post 142 and track 144, are shown in FIGS. 7A-7C, and other components, though not visible, are operating in accordance with the embodiments disclosed herein. Both the top 146 and bottom 148 portions of the track are connected in such a way that they form a single continuous track. The track 144, as shown in FIGS. 4 and 7A-7C, is recessed and has a smooth interior for receiving cylindrical tracking pins 350.

As the reset element 300 is displaced from its position in FIG. 7A to its position in FIG. 7B, its displacement has both a longitudinal and transverse component. The longitudinal component is parallel to the receiving direction 132, and is directed by the two constraint units within the carriage housing 120. The transverse component is perpendicular to the receiving direction 132, and is directed by the top portion 146 of the track. Thus, the combination of the constraint units and the top portion 146 of the track cause the reset element 300 to trace out a curved path 152 during the reset process of FIGS. 1A-1B, while the plane of the top portion 300a of the reset element remains normal with respect to the receiving direction 132.

As the reset element 300 is displaced from its position in FIG. 7B to its position in FIG. 7C, its displacement has a longitudinal component that is parallel to the receiving direction 132. The longitudinal component is directed by the two constraint units within the carriage housing 120. The reset element 300 is now engaged with the bottom portion 148 of the track, which extends along the receiving direction 132. Thus, the combination of the constraint units and the bottom portion 148 of the track cause the reset element 300 to trace out a longitudinal path parallel to the longitudinal axis 132 during the reset process of FIGS. 1B-1C, while the plane of the top portion 300a of the reset element remains normal with respect to the receiving direction 132.

The foregoing is merely illustrative of the principles of the disclosure, and the systems, devices, and methods can be practiced by other than the described embodiments, which are presented for purposes of illustration and not of limitation. It is to be understood that the systems, devices, and methods disclosed herein, while shown for use in recharging an actuated auto-injector, may be applied to systems, devices, and methods to be used in other devices that may require simultaneous adjustment and resetting of movable components.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. A device for resetting an auto-injector, the device comprising:
a housing having a receiving shaft into which the auto-injector can be inserted in a receiving direction;
a reset element configured to engage a collar of the auto-injector when the auto-injector is inserted into the receiving shaft, wherein the reset element moves the collar relative to the auto-injector in a first direction along the receiving direction; and
a post configured to engage a plunger within the auto-injector when the auto-injector is inserted into the receiving shaft, wherein the post moves the plunger in a second direction relative to the auto-injector opposite the receiving direction.

2. The device of claim 1, wherein the reset element comprises:
a frame; and
at least two elongate members extending from the frame and defining an opening therebetween;
wherein an inner ledge extends along the frame and the at least two elongate members about the opening, the inner ledge configured to couple with the collar.

3. The device of claim 2, wherein a portion of the inner ledge is tapered to form a wedge.

4. The device of any of claims 1, further comprising a base, wherein the post is connected to the base.

5. The device of claim 4, further comprising a track, wherein the track is connected to the base and the reset element engages the track.

6. The device of claim 5, wherein the reset element is displaced along the track.

7. The device of claim 5, wherein the reset element moves the collar relative to the auto-injector as the reset element is displaced along a first portion of the track.

8. The device of claim 7, wherein the reset element fixes the position of the collar relative to the auto-injector as the reset element is displaced along a second portion of the track different than the first portion of the track.

9. The device of claim 6, wherein a plane defined by a portion of the reset element that engages the collar remains normal to the receiving direction as the reset element is displaced along the track.

10. The device of claim 4, wherein the housing is configured to move relative to the base along the receiving direction in response to the reset element engaging the collar.

11. The device of claim 1, wherein the post is shaped to fit a complementary feature on the plunger.

12. A method for resetting an auto-injector, the method comprising:
receiving the auto-injector in a receiving direction;
engaging a collar of the auto-injector with a reset element, wherein the reset element moves the collar relative to the auto-injector in the receiving direction; and
engaging a plunger within the auto-injector with a post, wherein the post moves the plunger relative to the auto-injector opposite the receiving direction.

13. The method of claim 12, further comprising displacing the reset element along a track.

14. The method of claim 13, wherein the reset element moves the collar relative to the auto-injector as the reset element is displaced along a first portion of the track.

15. The method of claim 14, wherein the reset element fixes the position of the collar relative to the auto-injector as the reset element is displaced along a second portion of the track.

16. The method of claim 13, wherein a plane defined by a portion of the reset element that engages the collar remains normal to the receiving direction as the reset element is displaced along the track.

17. The method of claim 12, wherein engaging the plunger with the post comprises fitting a portion of the post to a complementary feature on the plunger.

18-29. (canceled)

30. A reset element comprising:
a frame portion; and
at least two elongate members extending from the frame portion and defining a first opening therebetween;
wherein an inner ledge extends along at least a portion of the frame portion and the at least two elongate members about the opening, the inner ledge configured to couple with an auto-injector.

31. The reset element of claim 30, wherein a portion of the inner ledge is tapered to form a wedge.

32. The reset element of claim 30, wherein the first opening defined by the at least two elongate members has a horseshoe shape.

33. The reset element of claim 30, wherein the frame defines a second opening that extends from a top portion of the frame to a bottom portion of the frame.

34. The reset element of claim 30, further comprising at least one tracking pin inside the opening and directed inward, the at least one tracking pin being configured to engage a separate track that passes through the opening.

35. The reset element of claim 30, further comprising at least one tracking pin that extends outward from the frame, the at least one tracking pin being configured to engage a separate track.

36. The reset element of claim 34, wherein the at least one tracking pin is cylindrically shaped.

37-44. (canceled)
